(19) United States
(12) Patent Application Publication
(10) Pub. No.: US 2016/0022810 A1
(43) Pub. Date: Jan. 28, 2016

(54) Method for Treating Cancers by Alternating Immunotherapeutics and Directly Oncolytic Therapeutics

(71) Applicant: David I. Cohen, Pelham, NY (US)

(72) Inventor: David I. Cohen, Pelham, NY (US)

(21) Appl. No.: 14/809,177

(22) Filed: Jul. 25, 2015

Related U.S. Application Data
(60) Provisional application No. 62/028,924, filed on Jul. 25, 2014.

(51) Int. Cl. A61K 39/39; A61K 31/675; A61K 51/10; A61K 47/48; A61K 31/7088; A61K 51/04; A61K 45/06; A61K 31/337

(52) U.S. Cl. CPC A61K 39/39; A61K 45/06; A61K 31/675; A61K 31/337; A61K 47/48384; A61K 31/7088; A61K 51/0491; A61K 51/1093; A61K 51/1045; A61K 2039/55516

(57) ABSTRACT

The present invention describes a unique immunotherapeutic method of treating cancer with the administration of CATS™ or an alternation of CATS™ immunotherapeutic followed by a directly oncolytic compound or agent, which protocol (BYES) can be by example an agent specifically targeting cancer stern cells, other cancer-specific growth pathways, radiotherapy, or compartmentalized chemotherapy. The BYES combination of CATS™ immunotherapeutic with cyclophosphamide delivers a very high statistical significant difference in survival and primary tumor control compared against either agent individually.

### RELATED APPLICATIONS

This application claims the benefit of U.S. Provisional Patent Application No 62/028,924, filed Jul. 25, 2014, the disclosure of which is incorporated herein by reference.

### FIELD OF THE INVENTION

The present invention relates to the field of combination therapeutics in the treatment of cancers, particularly for inducing long-term remissions or cures of advanced metastatic cancers. Specific realizations of this invention are combinations between innate immunotherapeutics, preferably Cysteine-rich Adjuvant Therapeutics (CATS), with an oncolytic chemotherapeutic or biologic. This strategy directs the oncoimmunologic to prepare the immune system for activity, immediately upon which fresh cancer targets are exposed to immune surveillance by the directed action of the oncolytic, as Bidirected oncolYtic Eradicators (BYES). A further preference in advanced and metastatic cancers is targeting the oncolytic drug against cancer stem cells to destroy the microenvironment nurturing cancer growth. Targeting of cancer stem cells can ideally and selectively be done through interruption of the WNT signaling pathway specific to each tumor phenotype, but also more generally by targeting against cancer (stem) cell surface proteins or receptors. The cancer oncolytic agent could also have activity against T Regulatory cells, but no claim is made here regarding the use of biologic T cell “checkpoint” inhibitors such as Yervoy®, either alone, in combination with PM checkpoint inhibitors, such as Opdivo® or Keytruda®, or in combination with the proprietary agents described in this invention.

### BACKGROUND

In the year 2000, an estimated 22 million people were suffering from cancers worldwide and 6.2 million deaths were attributed to these diseases. Every year, there are over 10 million new cases and this estimate is expected to grow by 50% over the next 15 years (WHO, World Cancer Report. Bernard W. Stewart and Paul Kleihues, eds. IARC Press, Lyon, 2003), Current cancer treatments are dominated by invasive surgery, radiation therapy and chemotherapy protocols, which are frequently ineffective and can have potentially severe side-effects, non-specific toxicity and/or cause traumatizing changes to an individual's body image and/or quality of life. One of the causes for the inadequacy of current cancer treatments is their lack of selectivity for affected tissues and cells. More selective cancer treatments would leave normal cells unharmed thus improving outcome, side-effect profile and quality of life.

Newly FDA-approved immunotherapeutics for metastatic melanoma, such as Yervoy®, Opdivo® or Keytruda®, are harbingers for treating a broad spectrum of human cancers by harnessing the natural protective power of the immune response. To date these approved “checkpoint inhibitors” are large biologic molecules that work by inhibiting T regulatory cells (TReg). Because of their size and complexity, they are frequently targets of immune autoreactivity with resultant toxicities, and more rarely life-threatening “cytokine storm.” Because TReg inhibitors are downstream regulators of immune response, they do not trigger fresh immune surveillance against tumors. In contrast agents capable of stimulating innate immunity deriving from dendritic cells, NK cells, or myelocytes, or countering innate suppression deriving from Tumor Associated Macrophage (TAMs) or Myeloid-derived Suppressor Cells (MDSCs), do support de novo anti-tumor immune responses. As an example of this class of oncoimmunologic, the anti-prostate cancer biologic Provenge® gained FDA-approval for the treatment of recurrent, refractory prostate cancer. As a cell-based therapy, however, Provenge® is expensive to manufacture, associated with small-batch variabilities, and has significant toxcities. New oncoimmunologic therapeutics in this class could deliver major advances in major cancer markets.

Breast and prostate cancers are among the most frequently diagnosed malignancies in the United States other than skin cancers. Generally these cancers can only be curatively resected, when detected early, and resection has little if any role in metastatic cancer. Non-surgical approaches, such as radiotherapy or chemotherapy, affect normal cells and result in side effects that limit treatment. Importantly, all current treatments for recurrent or metastatic cancer are only palliative. Consequently, development of novel systemic approaches to treat advanced, recurrent and metastatic cancers are urgently needed, particularly insofar as these approaches offer an extended quality of life to the diseased individual.

Immunotherapy has potential as a promising treatment for cancer patients because of its specificity and freedom from many of the toxic effects of chemotherapies. Cancer is one of many common human diseases that respond to immune-based treatments even at advanced stages. Clinical trials in humans have established that an immune response could regress some metastatic human melanomas, prostate cancers, lung cancers, and renal cancers. No oncoimmunologics are currently FDA-approved for the treatment of breast cancers. These observations were broadened by the discovery the dendritic cells, a specific class of antigen-presenting cells (APC), are particularly effective at initiating cytotoxic T lymphocyte (CTL) activity against cancers and other diseases. Technologies that target and activate dendritic cells have yielded some early successes against human prostate cancers and cervical premalignancies, caused by infection with Human Papilloma Virus (HPV).

Many current cancer immunotherapy strategies focus priming CTLs to lyse tumor cells. These antigen-specific cancer “vaccines” identify epitopes expressed by cancer cells that can be used as targets. However, the resident CTL epitopes of cancers are not always optimal because the CTL repertoire against highly expressed epitopes is often tolerized (Gross et al., J. Clin. Invest. 113(3):425-433, 2004).

The development of immunotherapeutic drugs to treat cancer has been hampered by technical difficulties in activating dendritic cells to deliver signals that overcome tolerance of CTL and other anti-cancer immune responses, Antigen targeting for the induction of a CTL response is a challenge insofar as natural processing requires that the antigen enter the cytoplasm of the cell in order to bind to the immune system's major histocompatibility complex (MHC) class I antigen, a prerequisite to CTL activation because the ligand for activating the T cell receptor on CTL is a complex of antigen and MHC class I. In almost all cases, protein antigens, even when they are coupled with a dendritic cells co-activator, enter exclusively into the alternative MHC Class II antigen presentation pathway that excludes CTL stimulation. This can be overcome, in part, by peptide-based technologies, because peptides bind to MHC Class I that is already on the surface of the dendritic cells. However, this technology is non-specific and most peptides are poor dendritic cells activators, which limits their efficacy as “vaccines” for human cancer.

While significant advancements have been made, treatment of cancers by chemotherapy frequently results in severe side effects because the therapy used is not specific to the cancer, killing non-cancerous cells including hematopoietic cells critical to immune surveillance. One important advantage as described here of preceding chemotherapy by immunotherapy is that immune stimulation is achieved before chemotherapeutic immunosuppression sets in, meaning that both agents work in an optimal sequence. When the cancer mutates to chemotherapy resistance, dosages must be increased, and the side effects become more pronounced. By restricting cancer replication via an oncoimmunologic, the potential for mutation to resistance decreases and the sensitivity of cancer cells to chemotherapy increases. The result is superior responses at tower dosages.

In addition to standard chemotherapy and hormone replacement therapy, new classes of therapies have emerged with directed oncolytic mechanisms. One approach targets either toxins or radioactive isotopes directly into the cancers by coupling the oncolytic agent to monoclonal antibodies (MAb) directed against cancer antigen. Genentech's Kadcyla® is an example of this kind of “smart-bomb” approved for the treatment of breast cancer. However, Kadcyla® is not an oncoimmunologic agent. Another class are drugs like Gleevac®(Novartis) that antagonize growth pathways specific to cancer cells, such as the ber-abl oncogene of chronic myelogenous leukemia targeted by Gleevac®. Other approaches are being designed directed against growth pathways specific to cancer stem cells, which are the seeds for cancer metastasis to distant sites. This stem cell strategy is a preferred realization of this invention.

At the present time, patients with recurrent cancer have few options of treatment that offer extended quality of life. The regimented approach to cancer therapy has produced overall improvements in global survival and morbidity rates. However, to the particular individual, these improved statistics do not necessarily correlate with an improvement in their personal situation. When cancer recurs after these consolidation therapies, it is almost always rapidly fatal even when treated by any of the newer targeted agents.

A superior approach to treatment would be a customized combination of stimulating the innate immune system from outside the cancer, and then inhibiting cancer-specific growth signaling mechanisms from inside the cancer cell. This one-two punch would first engage dendritic cells for cancer immunosurveillance, and then oncolysis by a chemotherapy or targeted agent would generate fresh cancer antigens to charge the dendritic cell almost like a customized cancer vaccine. According to the mechanism it is critical that the immunotherapy be administered first, because only this sequence primes the immune system before suppression by the oncolytic agent.

### SUMMARY

The present invention describes methods in the treatment of cancer that incorporates the administration of Cysteine-rich Adjuvant Therapeutics (CATS™) to activate the immune system and provide an immunotherapeutic means to treat cancer. One embodiment of the present invention provides for an alternating combination of CATS™ with a cytotoxic agent. Cyclophosphamide is a preferred realization of this combination strategy because cyclophosphamide, in addition to its chemotherapeutic efficacy against breast cancers, also antagonizes TReg immune suppression. Still another approach of the present invention alternates CATS™ with other targeting agents such as a targeted growth factor pathway inhibition using Directed Antagonists to Growth (DAGR™s) such as, but not limited to, RNAi, MAb coupled to a toxin or radioisotope, a directed antagonist to a cancer-specific growth pathway such as Gleevac®, or an anti-growth activity intrinsically linked into CATS™ so that CATS™ and DAGR™s are delivered sequentially as the biologic first binds to the cell surface and then enters the cell (cytoplasm and nucleus). Such Bidirected oncolYtic Eradicators (BYES) achieve superior remissions of advanced metastatic breast and other cancers, as demonstrated in the figures. Another embodiment incorporates targeted chemotherapeutic agents linked to a monoclonal antibody in combination with CATS™. In all these realizations, CATS™ is preferentially delivered prior to the DAGRs so that the tumor antigens shed by the lysed cancer cells can be rapidly attracted into the activated dendritic cell microenvironment.

### DESCRIPTION OF THE FIGURES

FIG. 1. depicts CATS™ activity in established breast cancer using an orthotopic murine model.

FIG. 2. depicts CATS™ activity in (pre-terminal breast cancer using an orthotopic murine model.

FIG. 3. Kaplan-Meier plot showing the survival benefit of CATS™ in pre-terminal breast cancer from an orthotopic murinebreast cancer trial.

FIG. 4. Graphical representation of sustainable activity with CATS™.

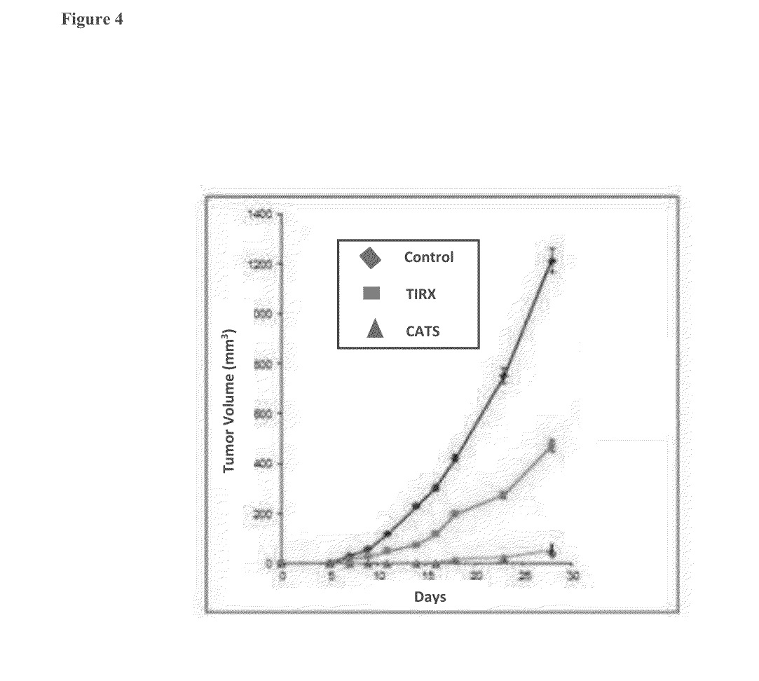

FIG. 5. Kaplan-Meier plot showing prolonged survival from CATS™ during advanced metastatic murine breast cancer.

FIG. 6. CATS™ inhibition of primary breast cancer growth compared to paclitaxel.

FIG. 7. CATS™ activity having a much stronger anti-metastatic activity than paclitaxel (taxol).

FIG. 8. Kaplan-Meier plot showing survival benefits of a single regimen using cyclophosphamide chemotherapy (CD) or CATS™ immunotherapy compared to untreated control (placebo).

FIG. 9. Primary murine breast tumor volumes showing long term depletion of primary tumor masses with alternating combination therapy of CATS™ immunotherapeutic and cytoxan chemotherapeutic.

FIG. 10. Kaplan-Meier plot showing long-term, stable remissions of advanced metastatic murine breast cancer using alternating combination therapy of CATS™ immunotherapeutic and cytoxan chemotherapy.

FIG. 11. Graph showing depletion of breast cancer stem cells by DAGRs squelching stem cell growth.

FIG. 12. Graph showing elimination of lung metastases in breast cancer depleted by DAGRs of stem cells.

### DETAILED DESCRIPTION OF THE INVENTION

Oncolmmunologics are targeted drugs aimed to controlling tumor growth and preventing or resolving metastases, while avoiding many of the side effects associated with standard chemotherapy. This is particularly relevant in breast cancer where disease strikes young women where loss of reproductive function and physical deformity are particularly disabling. Although many standard chemotherapies have efficacy against early breast cancer, only a few agents (eg. Paclitaxel are active in recurrent disease. Accept in unusual circumstances, such endstage breast cancer therapeutics give transient palliation. None of the currently approved oncoimmunologics are active in breast cancers

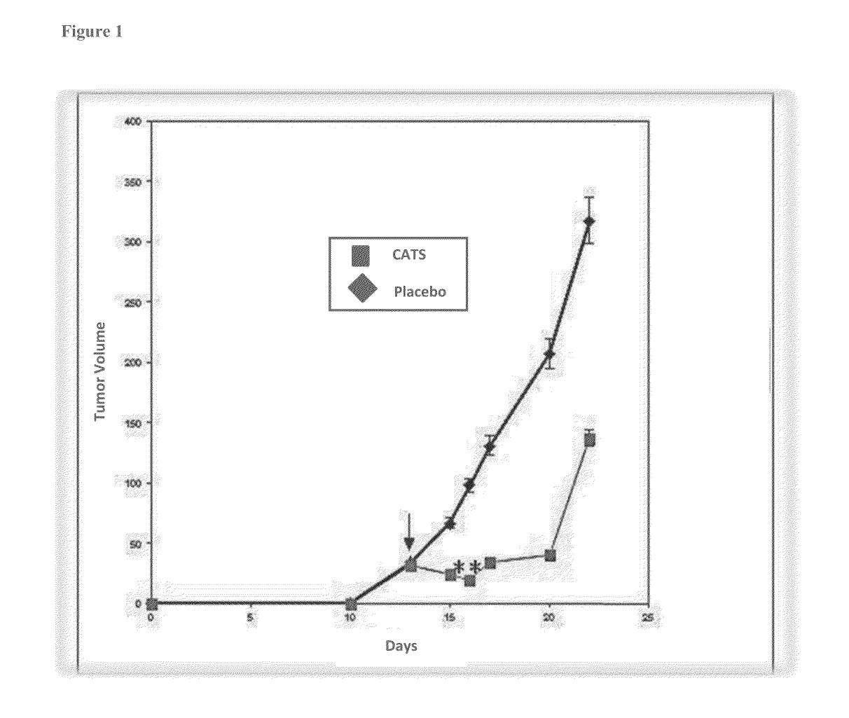

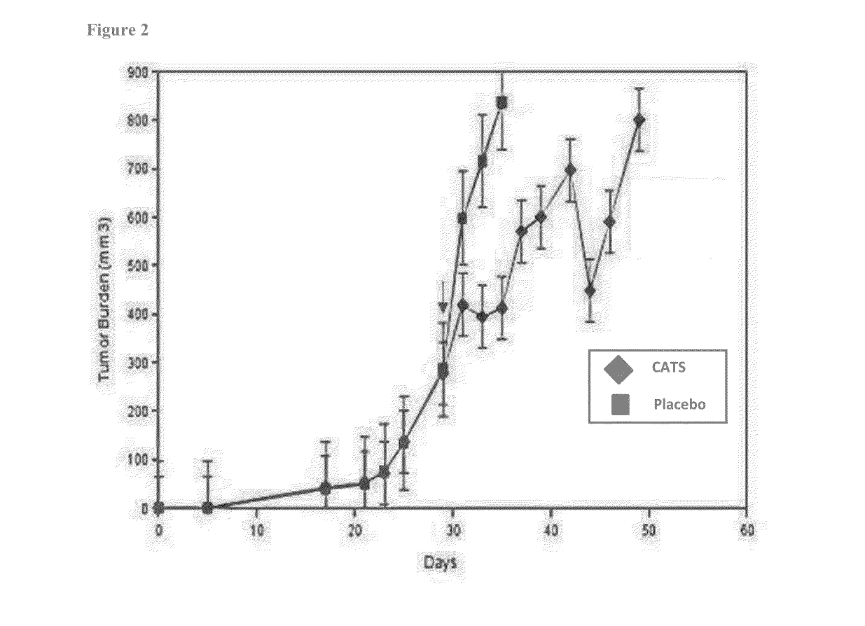

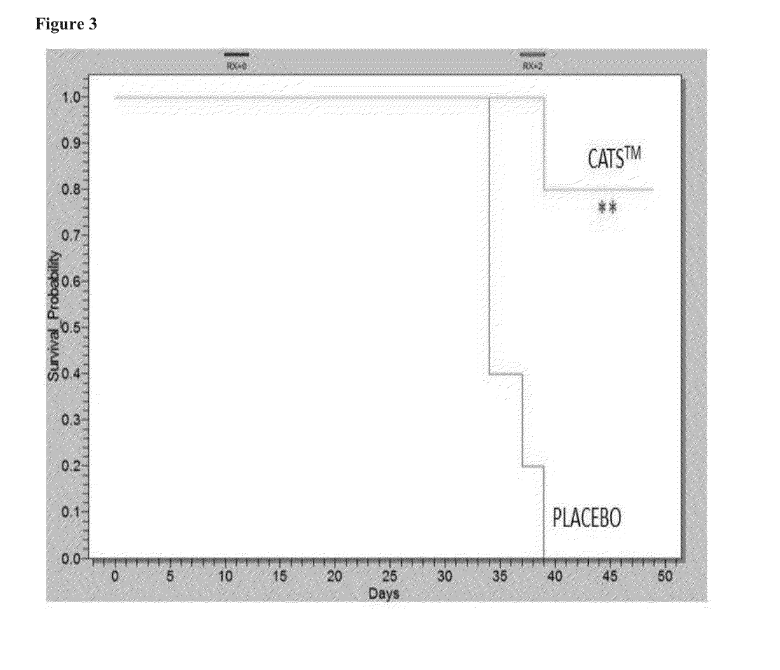

One embodiment of present invention incorporates the administration of Cysteine-rich Adjuvant Therapeutics (CATS™) in the treatment of tumors and reducing tumor growth. Using an orthotopic murine model for established breast cancer, significant reduction in the size of primary tumors was obtained in mice receiving CATS™. FIG. 1 depicts CATS™ ability to reduce the size of a primary tumor by day 16 in an orthotopic murine model for established breast cancer. In this model, two cohorts of 10 BALB/c mice each subcutaneously implanted in the mammary pad with 10⁴ 4T1 breast cancer cells on day 0. One group was dosed once every week with CATS™ at 25 nm per mouse intravenously for three weeks beginning at day 12. The control group was treated with 25 ng of an inactive protein using the same doing regimen (placebo). The size of the primary tumor was determined using a standard formula (length×width×0.72). By day 16 the reduced size of the primary tumors in CATS™ treated mice were significantly smaller than controls and remained smaller throughout the duration of the trial (p<0.01**), Mice were sacrificed when tumor diameter reached 15 min. This treatment albeit short lived provided a significant reduction in tumor size throughout the duration of the trial. This is further exemplified in advanced disease. Shown in FIG. 2 is CATS™ activity in pre-terminal breast cancer using the 4T1 orthotopic murine model. Following orthotopic implant of 4T1 cells into Balb/c mammary fat pads, treatment was delayed 28 days until tumor had grown to an average size of 300 mm³, and a matched cohort of mice averaged 25 visible pulmonary metastases. At days 28 and 30 (consolidation phase), and weekly thereafter, mice were intravenously administered either 25 ng CATS™, or 25 ng inactive protein control (Placebo). Primary tumor size was then recorded, With 10 mice in each cohort, there was a significant decrease in primary tumor burden with CATS™ administration (p<0.05). When the same orthotopic murine model was used to assess survival, a Kaplan-Meier plot of survival for each group showed good, survival benefit through CATS™ treatment (see FIG. 3). All mice in the placebo group were dead by day 39, while 8 of 10 CATS™-treated mice survived until day 50, which is highly statistically significant (p<0.005 **). No mice in either group survived beyond day 51.

Another embodiment of the present invention is sustained treatment of breast cancer by CATS™ CATS™ differ from Tat-based immunotherapeutics (TIRX or “PINS”) described earlier by my laboratory, insofar as CATS™ are fully human biologics devoid of immunosuppressive toxicities associated with TIRX or “PINS”. Mice (ten per cohort) were implanted s.c. in the mammary fat pad on Day 0 with 1×10⁵ 4T1 breast cancer cells (ten times our standard 4T1 implantation), and then treated biweekly starting on Day 4 with 10 ng IV inactive protein (Control, Blue), TIRX (Magenta) or CATS (Green) until further dosing was no longer tolerated. Over the course of this protocol mice received 8 injections of CATS over tour weeks, white 8/10 mice receiving TIRX in a parallel trial succumbed acutely to their fifth TIRX injection, presumably by cytokine storm, so that in the therapeutic trial shown 4 doses of TIRX (weeks one and two) were administered. 1⁰ tumor volume is recorded as ((length (mm)×width (mm)²)×0.52). The difference in 1⁰ growth suppression by CATS over TIRX is highly statistically significant (P<0.01) even at the end of two weeks when TIRX therapy was forced to cease. At day 28 when all animals were sacrificed, 4/10 CATs-treated animals were in complete remission, as determined by scarified or absent primary tumor, and no visible lung metastases.

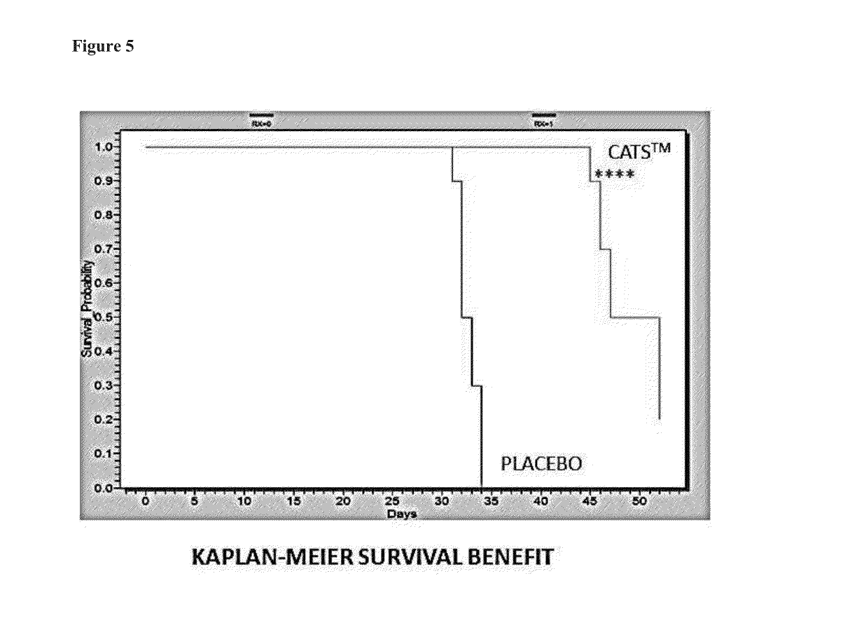

CATS™ provided survival benefit in endstage murine breast cancer (see FIG. 5). Ten mice per cohort with 4T1 breast cancer orthotopically implanted 25 days previously were intravenously treated with two boluses of 25 ng CATS™, separated by 48 hours, or with an identical regimen of 25 ng inactive protein (PLACEBO). In a simultaneously matched cohort sacrificed at day 25, all of the mice (100%) had extensive lung metastases (range 48-370 by clonogenic assay), The CATS™ cohort had 9 out of 10 mice alive at day 46, while in the control group 0 out of 10 mice survived past day 34, a very highly statistical significance (p=0.0001****). Five of 10 mice in the CATS™ treatment group surviving at Day 51 were administered one additional CATS™ bolus (25 ng iv), and two mice entered long-term remissions extending past 90 days (not shown).

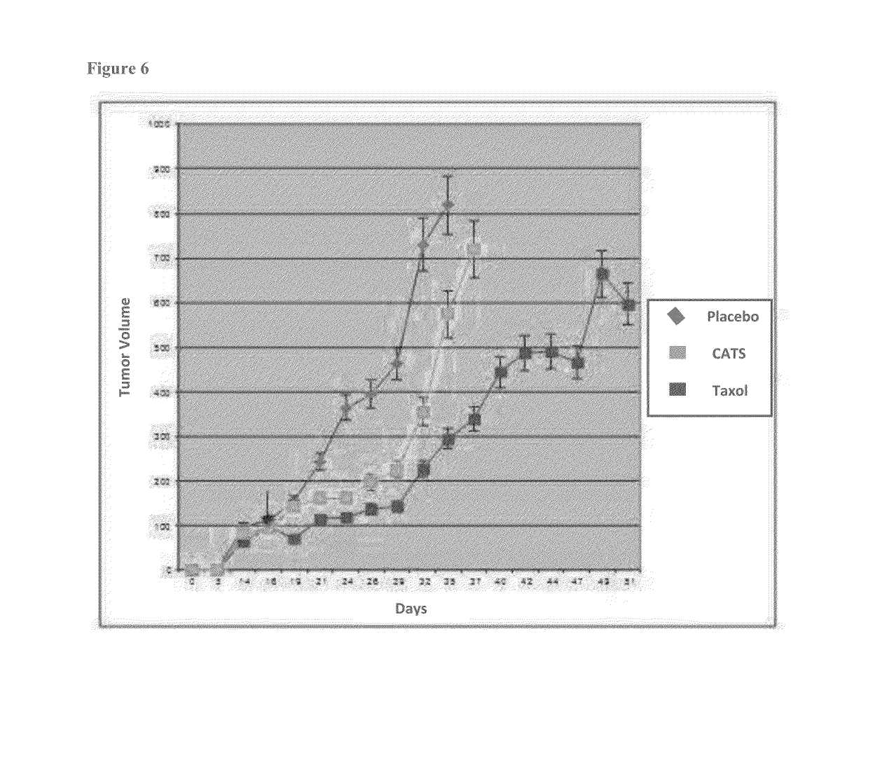

FIG. 6 depicts the efficacy of CATS™ compared to paclitaxel in reducing primary tumors growth. Three groups of 10 BALB/c mice were implanted orthotopically with 5×10⁴ 4T1 breast cancer cells. Each of 10 mice in the CATS™ treatment group (CATS™, blue squares) received 25 ng CATS™, beginning on day 16 (arrow) when tumor volume had reached 100 mm³, and thereafter once weekly. Each of ten mice in the control cohort (Placebo, red triangles) received 25 ng of inactive protein. A third cohort of ten mice was injected intraperitoneal (ip) with Taxol 100 micrograms per mouse. The results in FIG. 6 are shown as mean tumor volume (mm³), calculated from the formula (length (mm)×width (mm)×0.52 (mm)) bars, ±SE. A significantly greater reduction in primary tumor volume of CATS™ compared to paclitaxel was observed (p<0.05).

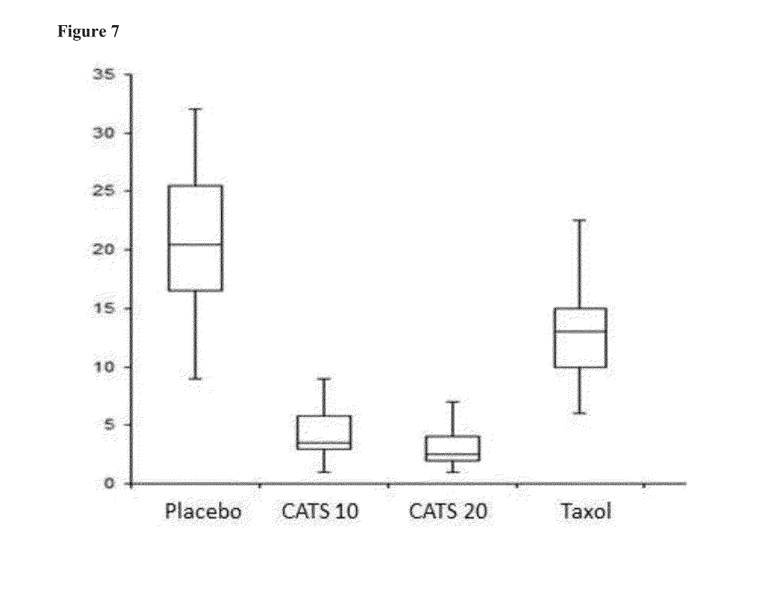

This reflects into a much stronger anti-metastatic activity (FIG. 7) of CATS™ over paclitaxel, that translates to survival benefits. In this study, 40 BALB/c mice were orthotopically implanted in the mammary pad on Day 0 with 1×10⁴ 4T1 breast cancer cells. When tumor volume approximated 200 mm³ (on Day 17), mice were randomly distributed into four cohorts of ten mice each. The control group (Placebo) received two boluses separated by 2 days (consolidation phase) of 20 ng inactive protein intravenously (iv), and thereafter weekly Placebo (20 ng), two CATS™ treatment cohorts received iv either 10 ng (CATS™) or 20 ng (CATS™20) consolidation and thereafter weekly, and the fourth cohort received 100 micrograms paclitaxel (Taxol) weekly (consolidation dosing with Taxol was toxic). Mice were sacrificed when tumor diameter reached 14 mm (Day 30), at which time mice were euthanized, lungs removed, and visible lung metastases were quantitated by a second observer blinded to the treatment protocols. Floating bars depict maximum and minimum number of metastases each cohort, horizontal line indicates mean±SE. The reduction in mean metastases in mice that were administered 20 ng CATS™ compared to 100 micrograms Taxol showed a very high statistical significant difference (p<0005).

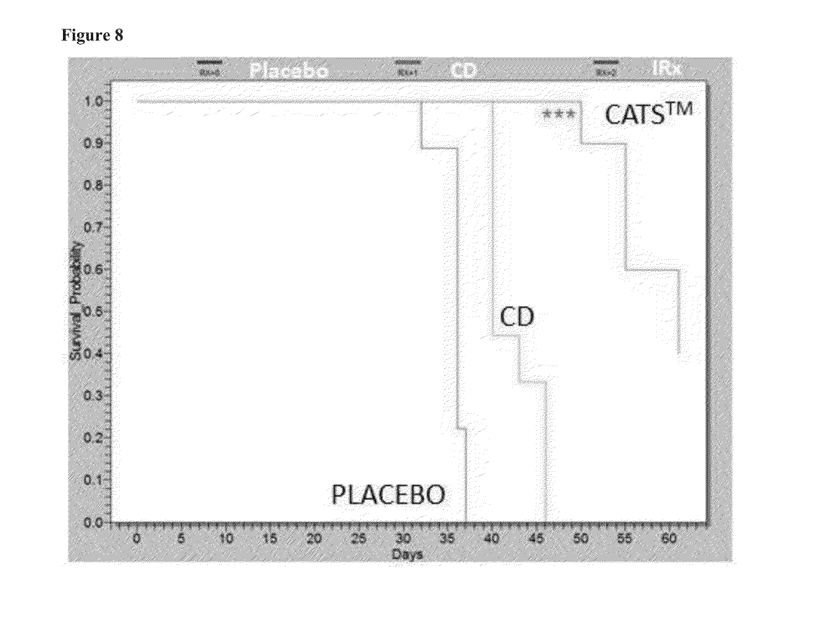

CATS™ improved survival as a single regimen significantly over cytotoxic drug (CD, Cyclophosphamide, FIG. 8). Thirty mice were orthotopically implanted with 5×10⁴ 4T1 breast cancer cells in their mammary fat pad on Day 0. On Day 16, mice were randomized into three cohorts of ten mice each to receive weekly doses of either carrier control (Brown line, Placebo, 25 ng iv), cyclophosphamide cytotoxic drug (CD) chemotherapy (Green line, CD, 80 mg/kg ip) or CATS™ immunotherapy (Purple line, 25 ng iv). Kaplan-Meier curves analyzing surviving mice in each cohort show no mice survived beyond 37 days in the control group, or 46 days in the CD group, while 4 of 10 mice were alive at day 62 in the CATS™ treatment group. This benefit of survival with CATS treatment had a very high statistical significance over control (p<0.001, ***).

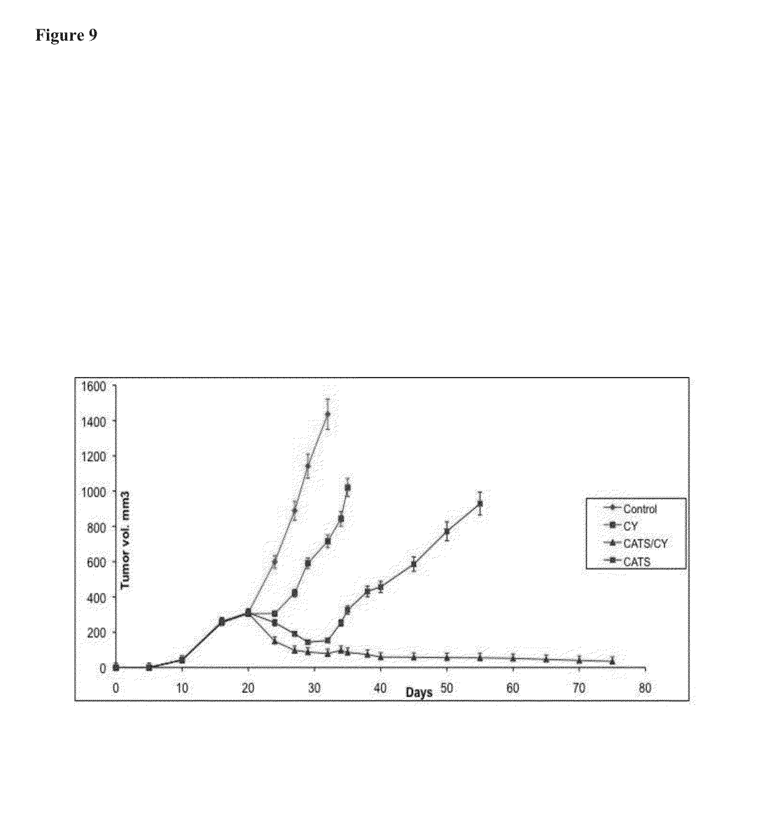

The ultimate strategy and invention proposed here is to alternate CATS™ immunotherapeutic with an oncolytic drug in a protocol that first primes and countersuppresses the immune system, and then releases fresh cancer antigens to trigger an oncoimmunologic response. The realization demonstrated in FIG. 9 is a regimen alternating CATS™ with cyclophosphamide, where cyclophosphamide has the additional activity to antagonize TReg suppression. Four groups of 10 BALB/c mice were implanted with 1×10⁴ 4T1 cells injected s.c into the mammary fat pad. Treatment was initiated on Day 20 when tumor diameters averaged 300 mm³. Mice were randomized to protocols initiated with two boluses spaced by two days, and thereafter weekly doses of placebo (Control, red, 50 ng/dose IV), cyclophosphamide (CY, green, 80 mg/kg ip), CATS™ (blue, 50 ng IV), or alternating once weekly dosages (purple) initiated with CATS™ (50 ng IV) and followed the next week with cyclophosphamide (80 mg/kg) in a cyclical manner. Tumor burden (tumor size mm³) was calculated using a standard formula The difference in mean tumor burden between the cohort receiving combination therapy and the cohort receiving CATS™ alone was highly statistically significant (p<0.001) by Day 40 and remained so throughout the duration of the trial.

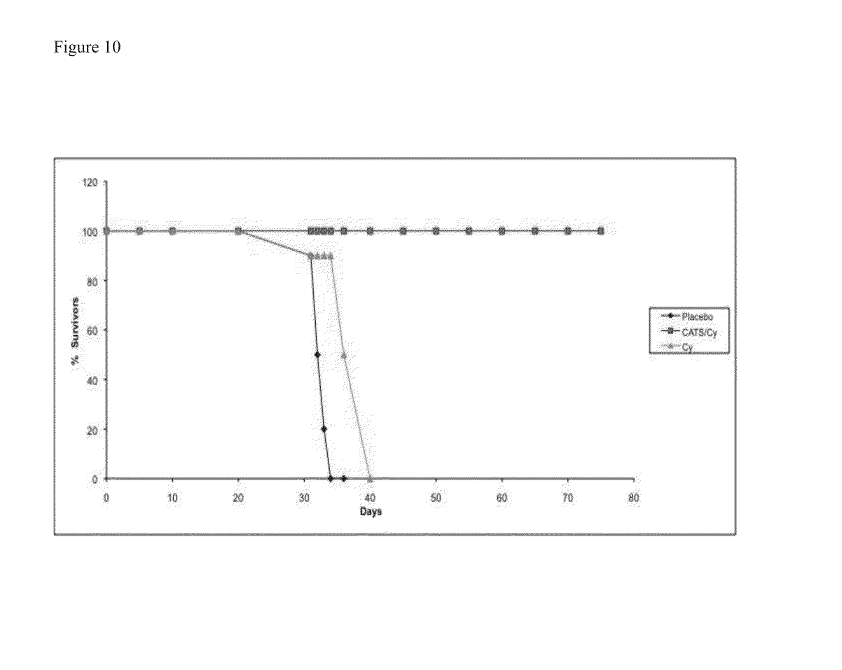

FIG. 10 demonstrates the survival benefit of CATS™ combination therapy. This Kaplan-Meier graph plots survival of three groups of mice (10/cohort) implanted with 1×10⁴ 4T1 cells on Day 0 and initiated to treatment on Day 20 with two bolus doses over the first 48 hours, and thereafter once weekly of either placebo (blue, 50 ng IV), cyclophosphamide (Cy, orange, 80 mg/kg ip) or alternating therapy (CATS/Cy, green, 50 ng IV CATS™ boluses or every other week, alternating every other week with Cy 80 mg/kg). In this trial 100% of mice treated with CATS™ alternating therapy survived 75 days, at which time the trial was terminated. The survival benefit of alternating CATS™ with Cy, over Cy treatment alone is very highly statistically significant (p<0.0001) at Day 40, when all of the mice receiving cyclophosphamide alone were dead.

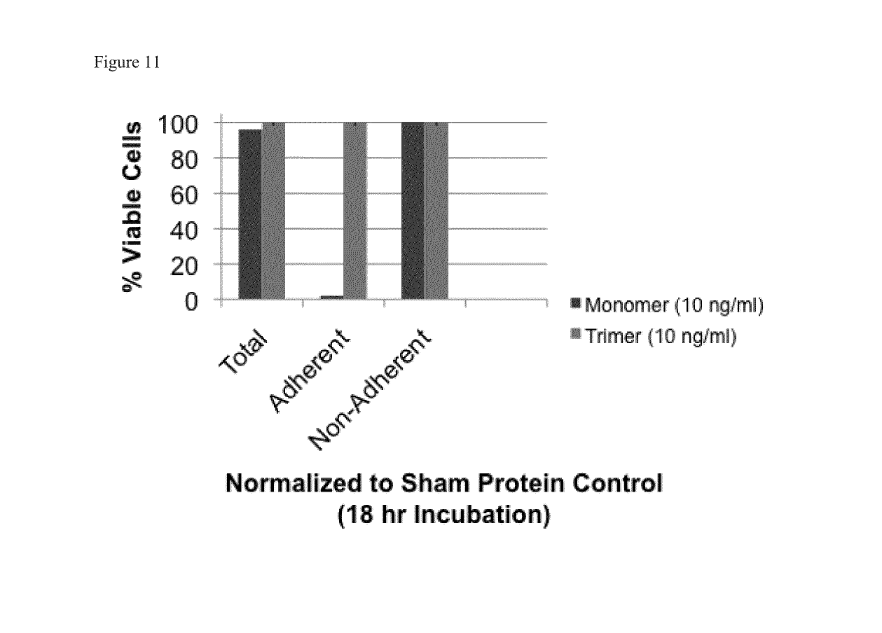

A therapeutic active in depleting stem cells could be particularly efficacious at reducing and treating metastatic disease. The 4T1 murine breast cancer cell line grows in vitro as a mixed cell population of adherent and non-adherent cells. Adherency is a well-recognized property of cancer stem cells. FIG. 11 demonstrates that adherent 4T1 (stem) cells are depleted by monomeric Tat. Cultures (100 ml) of the murine breast cancer cell line 4T1 were expanded in Falcon® 250 ml tissue culture flasks in RPMI 1640 medium and 10% PCS until reaching concentrations of approximately 2×10⁶ cells/ml, according to protocols recommended by ATCC. Under these conditions 4T1 expands as a mixed population of a few adherent cells that are mainly outgrown by cells released from the plastic and growing free in suspension. Total 4T1 (Total, left bars) cells were harvested by scraping the flasks, pooling cells, and centrifugation.

Non-Adherent cells (Right bars) were harvested by decanting the flasks, in which case remaining 4T1 cells are clearly visible adhering to the flasks. Such Adherent 4T1 cells (middle bars), typically constituting 1-2% of the 4T1 population, are covered with 10 ml fresh RPMI 1640 and scraped into the RPMI. After centrifugation, 4T1 cells were resuspended at 1×10⁶ cells/ml and treated (10 ng/ml protein) for eighteen hours @37° C. with either Sham protein, which is monomer Tat deleted at its amino terminus, Monomer Tat, or Trimeric Tat all synthesized in E. Coli. Viable cells were enumerated through trypan blue exclusion, and % viable cells scored as the ratio of viable treated cells/viable sham-treated cells to normalize for non-specific cell death (about 5% of cells) due to overnight culture.

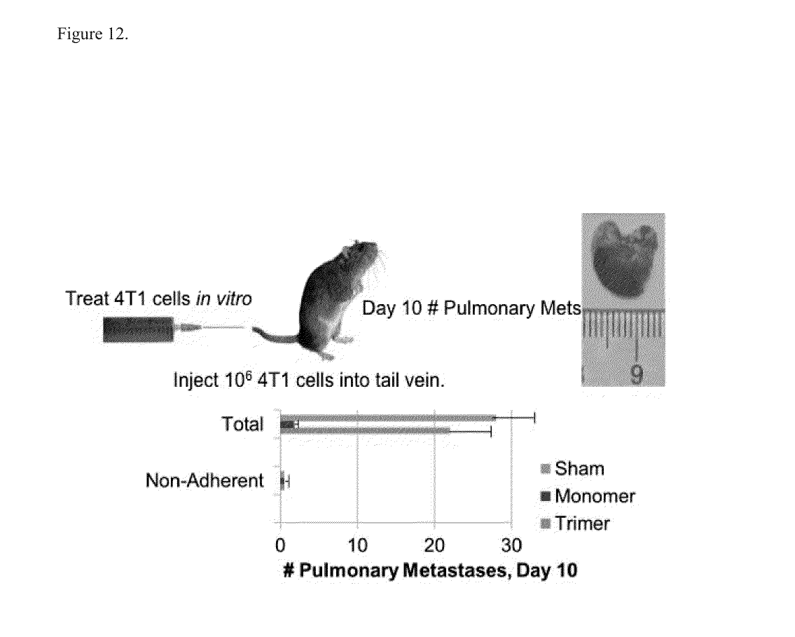

An embodiment of the strategy of depleting stern (adherent) cells to reduce metastatic cancer spread is illustrated in FIG. 12. As previously, cultures (100 ml) of the murine breast cancer cell line 4T1 were expanded in Falcon® 250 ml tissue culture flasks in RPMI 1640 medium and 10% FCS until reaching concentrations of approximately 2×10⁶ cells/ml, according to protocols recommended by ATCC. Total 4T1 (Total) cells were harvested by scraping the flasks, pooling cells, and centrifugation. Non-adherent cells were harvested by decanting the flasks. In the non-adherent pool, the remaining 4T1 cells that are clearly visible adhering to the flasks are discarded After centrifugation, 4T1 cells were resuspended at 1×10⁷ cells/ml and treated (100 ng/ml protein) for one hour @37° C. with either Sham protein, which is monomer Tat deleted at its amino terminus, Monomer Tat, or Trimeric Tat all synthesized in E. Coli. Following this incubation, 4T1 cells were washed 3 times, resuspended at 1×10⁷ cells/ml, and 5 mice/cohort each received 0.1 ml of cell suspension via tail vein injection. 10 days later mice were sacrificed, and visible pulmonary metastases quantified by two observers. The average number (#) of pulmonary metastases at Day 10 for each cohort is recorded in the bar graph, with standard error indicated by the flag.

Other embodiments of the present invention incorporate the combination of CATS™ with other known treatment regimens such as, but not limited to, radiation therapy, targeted growth factor pathway inhibition (DAGR™s=Directed Antagonists to Growth) using, for example, RNAi, or targeted chemotherapy such as with a chemotherapeutic linked to a monoclonal antibody. A preferred realization is a DAGR™ that targets and kills stem cells, as illustrated in FIGS. 11 and 12. It is also contemplated that DAGR™s can be an FDA approved agent (i.e. a chemo Rx) locally targeted either by IP injection, intravenous administration to the active site, or by proximal administration to the active site such as, but not limited to, intrapleural administration for lung metastasis. it is further contemplated that CATS™ can also be administered intrapleural for lung metastasis. Although the present invention has been described with reference to specific embodiments, workers skilled in the art will recognize that many variations may be made therefrom and it is to be understood and appreciated that the disclosures in accordance with the invention show only some preferred embodiments and advantages of the invention without departing from the broader scope and spirit of the invention. It is to be understood and appreciated that these discoveries in accordance with this invention are only those which are illustrated of the many additional potential applications that may be envisioned by one of ordinary skill in the art, and thus are not in any way intended to be limiting of the invention. Accordingly, other objects and advantages of the invention will be apparent to those skilled in the art from the detailed description together with the claims.

What is claimed is:

1. A method for treating cancer in a patient comprising:
a. administering a therapeutically effective amount of a Cysteine-rich Adjuvant;
b. causing a cessation of growth or regression of said cancer in said patient.

2. The method of claim 1 wherein said administering is initiated with a bolus dosing to arrest tumor progression.

3. The method of claim 1 wherein the metabolic breakdown of the Cysteine-rich Adjuvant is delayed by genetic modifications or other means, producing a sustained action.

4. The method of claim 1 wherein said cancer is endstage.

5. A method for treating cancer in a patient comprising:
a. Administering a therapeutically effective dose of an immunotherapeutic thereby activating and priming the immune system to be on alert to fight the cancer;
b. Administering a second therapeutically effective dose of an oncolytic agent to kill the cancer cells causing them to shed cancer antigens;
c. Causing a cessation of growth or regression or cure of said cancer in said patient.

6. The method of claim 5 where the immunotherapeutic is a Cysteine-rich Adjuvant, and the oncolytic agent is cyclophosphamide, the oncolytic having an additional activity to antagonize TReg immune suppression.

7. The method of claim 5 where said cancer is at an advanced stage and/or extensively metastatic.

8. The method of claim 5 (b) where the oncolytic agent induces the cancer cell to undergo apoptosis.

9. The method of claim 7 where the pro-apoptotic oncolytic cancer agent is a targeted growth inhibitor.

10. The method of claim 8 wherein the targeted growth inhibitor is paclitaxel coupled to a monoclonal antibody, and the cancer is breast or ovarian cancer.

11. The method of claim 8 wherein the targeted growth inhibitor is a directed antagonist to growth (DAGR), which can be targeted to inhibit a tumor-specific growth pathway such as a growth pathway in cancer stem cells.

12. The method of claim 8 where the cancer is chronic myelogenous leukemia and the DAGR is directed against ber-abl.

13. The method of claim 10 wherein the directed antagonist to growth is RNAi.

14. The method of claim 8 wherein the targeted growth inhibitor is radiation therapy using a radionucleotide.

15. The method of claim 12 where radionucleotide is coupled to an anti-cancer MAb

16. The method of claim 12 where locally administered radiation is alternated with systemic immunotherapy delivered either percutaneously or via intravascular catheter

17. The method of claim 8 where targeting is achieved by compartmentalized delivery of an FDA approved cancer chemotherapy alternated with either systemic immunotherapy or compartmentalized immunotherapy.

18. The method of claim 15 where delivery is Intrathecal administration for the treatment of brain cancer or brain metastases, Intrapleural administration for the treatment of lung cancer or lung metastases, or Intraperitoneal administration for the treatment of liver cancer, pancreatic cancer, ovarian cancer, or other cancers.

19. A method of claim 8 where targeting is achieved by compartmentalized delivery of a novel antagonist to cancer growth alternated with either systemic immunotherapy or compartmentalized immunotherapy.

20. The method of claim 17 where delivery is Intrathecal administration for the treatment of brain cancer or brain metastases, Intrapleural administration for the treatment of lung cancer or lung metastases, or Intraperitoneal administration for the treatment of liver cancer, pancreatic cancer, ovarian cancer, or other cancers.

21. A method of implanting a device that delivers slow release immunotherapy in alternation with slow release chemotherapy.

22. The method of claim 19 where the device is regulated by external control.
